(19) United States
(12) Patent Application Publication
(10) Pub. No.: US 2004/0120901 A1
(43) Pub. Date: Jun. 24, 2004

(54) Dental compositions including enzymes and methods

(76) Inventors: Dong Wu, Woodbury, MN (US); Joel D. Oxman, Minneapolis, MN (US); Sumita B. Mitra, West St. Paul, MN (US); Ingo Reinhold Haberlein, Weilheim (DE)

Correspondence Address: 3M INNOVATIVE PROPERTIES COMPANY, PO BOX 33427, ST. PAUL, MN, 55133-3427, US

(21) Appl. No.: 10/327,411

(22) Filed: Dec. 20, 2002

(51) Int. Cl. A61K 7/28; A61C 5/00

(52) U.S. Cl. 424/50; 433/217.1

(57) ABSTRACT

A hardenable dental composition that includes a polymerizable component and a therapeutic enzyme mixed within the polymerizable component, wherein upon hardening the polymerizable component to form a hardened dental material having a therapeutic enzyme mixed therein, the hardened dental material with the enzyme mixed therein is in contact with saliva in a subject's mouth for at least 1 day.

### BACKGROUND

Enzymes have been used in products for the improvement of oral health. Such products include, for example, mouthwashes, toothpastes, dentrifices, and the like. For example, the enzyme glucose oxidase has been used in mouthwash products to reduce halitosis. Also, a plaque hydrolyzing enzyme called DXAMase is proposed for use in toothpastes and mouthwashes. Enzymes have also been proposed for use in a microstructured delivery device for the oral environment.

These products are used for short periods of time (e.g., often for minutes or hours, and generally for less than one day) and are treated as disposables and discarded after each application. Thus, what is needed are compositions that include enzymes that can provide a desirable effect over an extended period of time.

### SUMMARY

The present invention is directed toward compositions containing enzymes and polymerizable components that can be used in a variety of dental applications. Such compositions are particularly useful for providing contact between saliva in a patient's mouth and an enzyme over an extended period of time. The enzyme is blended into the composition and upon hardening is mixed throughout the hardened material and is available at the surface of the hardened material. These compositions can be used in methods and for improving the oral health of a subject having the dental material incorporated into their mouth for an extended period of time.

More specifically, the present invention provides a dental composition that includes a polymerizable component and a therapeutic enzyme mixed within the polymerizable component, wherein upon hardening the polymerizable component to form a hardened dental material having a therapeutic enzyme mixed therein, the hardened dental material with the enzyme mixed therein is in contact with saliva in a subject's mouth for at least 1 day (preferably, for at least 7 days).

In a preferred embodiment, the present invention provides a hardenable dental composition that includes a polymerizable component and a therapeutic enzyme mixed within the polymerizable component; wherein upon hardening the polymerizable component to form a hardened dental material having a therapeutic enzyme mixed therein, the hardened dental material with the enzyme mixed therein is in contact with saliva in a subject's mouth for at least 1 day; wherein the therapeutic enzyme is selected from the group consisting of an oxidoreductase, a hydrolase, and combinations thereof; and wherein the polymerizable component is selected from the group consisting of epoxy resins, vinyl ether resins, ethylenically unsaturated compounds, glass ionomer cements, and combinations thereof.

In another preferred embodiment, the present invention provides a hardenable dental composition that includes a polymerizable component and a therapeutic enzyme mixed within the polymerizable component; wherein upon hardening the polymerizable component to form a hardened dental material having a therapeutic enzyme mixed therein, the hardened dental material with the enzyme mixed therein is in contact with saliva in a subject's mouth for at least 1 day; wherein the therapeutic enzyme is selected from the group consisting of oxidases, peroxidases, laccases, proteases, carbohydrases, lipases, and combinations thereof; and wherein the polymerizable component is selected from the group consisting of (meth)acrylates, (meth)acrylamides, and combinations thereof.

The present invention also provides methods. In one embodiment, the present invention provides a method of delivering a therapeutic enzyme to a subject's mouth to improve the oral health of the subject. The method includes: providing a hardenable dental composition that includes a polymerizable component and a therapeutic enzyme mixed within the polymerizable component; placing the hardenable dental composition in the mouth of the subject; and hardening the composition to form a hardened dental material having a therapeutic enzyme mixed therein, the hardened material with the enzyme mixed therein is in contact with saliva in a subject's mouth for at least 1 day.

### DETAILED DESCRIPTION OF PREFERRED EMBODIMENTS

The present invention is directed toward compositions containing enzymes and polymerizable components that can be used in a variety of dental applications. Such compositions are particularly useful for providing a therapeutic enzyme to the surface of a hardened dental material over an extended period of time. These compositions can be used in methods and kits for improving the oral health of a subject having the dental material incorporated into their mouth such that the hardened dental material with the enzyme mixed therein is in contact with saliva in a patient's mouth for an extended period of time (e.g., for at least 1 day, alternatively at least 7 days, and typically much longer such as at least 30 days, and often at least 90 days).

The hardened dental material can be any of a wide variety of materials that are prepared from polymerizable materials. Preferably, however, the hardened dental material is not a surface pre-treatment material (e.g., etchant, primer, bonding agent). Rather, preferably, the hardened dental material is a restorative (e.g., composite, filling material or prosthesis), sealant, coating, cement, or orthodontic adhesive.

Thus, the present invention provides materials that can preferably provide extended contact between the enzyme and the environment of the mouth. Enzymes that remain biologically active over the contact time (or for a significant portion thereof) are particularly desirable. That is, preferably, the present invention provides an enzyme mixed within a hardened material that is available for reaction with an enzyme substrate (whether it be in the mouth and/or provided in the hardened material itself) to provide beneficial results. Prior to the present invention it was unexpected that an enzyme could be incorporated into a hardenable dental composition, which upon hardening provides exposure of the enzyme to the oral environment for an extended period of time.

Such compositions can prove to be very valuable and beneficial compositions for dental care. Typically, the use of enzymes instead of chemicals can be less harmful to a patient's health and cause fewer side effects. This is particularly true for the prevention of dental plaque, which contributes to dental tooth and gum diseases. Enzymes useful in the practice of this invention are therapeutic enzymes, which herein means that they cause (e.g., by catalysis) the decomposition of harmful carbohydrates, proteins, lipids, and/or bacterial substrates in the mouth of a subject (e.g., in the oral plaque and saliva). A preferred group of enzymes generate bactericidal products (e.g., H₂O₂).

Optionally, one or more enzyme substrates can be added to the compositions of the present invention to enhance the therapeutic function of the enzymes. For example, for systems requiring water for activation, an enzyme substrate could be present in the composition with the enzymes if the system is anhydrous until use, thereby keeping the enzymes and substrate from interacting. Alternatively, the enzymes and corresponding enzyme substrates can be packaged in separate parts of a kit to keep them from interacting until desired.

It is advantageous to use enzymes that are substantially active at a pH prevailing in the mouth. Typically, this is about pH 5.0 to about pH 9.0, more typically about pH 6.0 to about pH 8.5, and even more typically about pH 6.4 to about pH 7.5. Under certain conditions the pH may be significantly lower, which allows for the use of a wider variety of enzymes.

Oxidoreductase and hydrolase enzymes are useful classes of enzymes for use in the present invention. Depending on the desired effect, any of the various types of enzymes can be preferred for a particular embodiment.

Oxidoreductase enzymes are those classified under the Enzyme Classification number E.C. 1 in accordance with the Recommendations (1992) of the International Union of Biochemistry and Molecular Biology (IUBMB). They catalyze oxidoreductions (i.e., redox reactions). Within the group of oxidoreductase enzymes are oxidase enzymes, peroxidase enzymes, and laccase enzymes.

Oxidase enzymes catalyze the oxidation of a substrate by acting on O₂ as an acceptor of electrons and forming hydrogen peroxide. Such enzymes are classified under the enzyme classification E.C. 1.1.3, E.C. 1.2.3, E.C. 1.3.3, E.C. 1.4.3, E.C. 1.5.3. E.C. 1.7.3, E.C. 1.8.3, E.C. 1.9.3. Examples include, but are not limited to, glucose oxidase, sucrose oxidase, lactate oxidase, (S)-2-hydroxy-acid oxidase, hexose oxidase, L- or D-amino-acid oxidase, xylitol oxidase, xanthine oxidase, glycolate oxidase, L-sorbose oxidase, alcohol oxidase, gulonolactone oxidase. Corresponding enzyme substrates include, but are not limited to, β-D-glucose, sucrose, lactate, (S)-2-hydroxy-acid, broad spectrum of carbohydrates including D-glucose, D-galactose, D-mannose, maltose, lactose, and cellobiose, etc., L-or D-amino acids, xylitol, xanthine, α-hydroxy acids, L-sorbose, a primary alcohol, and L-gulono-1,4-lactone.

Peroxidase enzymes act on peroxide as an acceptor of electrons. These include enzymes classified under the enzyme classification E.C. 1.11. The different types of peroxidase enzymes are distinguished by the donor molecules from which they take electrons to donate to hydrogen peroxide. In accordance with the present invention a peroxidase is used to generate free radicals from donor molecules. The donor molecules are typically capable of acting as a substrate for the peroxidase in generating such free radicals. Examples include, but are not limited to, horseradish peroxidase, soybean peroxidase, polyphenol peroxidase, manganese peroxidase, L-ascorbate peroxidase, chloroperoxidase, and iodide peroxidase. Corresponding enzyme substrates include, but are not limited to, hydrogen peroxide and electron donor molecules such as polyphenol, manganese (II), ascorbic acid, chloride, and iodide.

Laccase enzymes act on O₂ and yield water without any need for peroxide. These include enzymes classified under the enzyme classification E.C. 1.10.3. Corresponding substrates include, but are not limited to, O- and P-quinols, aminophenols, and phenylenediamine.

Hydrolase enzymes are those classified under the Enzyme Classification number E.C. 3 in accordance with the Recommendations (1992) of the International Union of Biochemistry and Molecular Biology (IUBMB). Within the group of hydrolase enzymes are protease enzymes, carbohydrase enzymes, and lipase enzymes. Preferred hydrolase enzymes are proteases.

Protease enzymes act to break down or hydrolyze proteins. Such enzymes are classified under the classification E.C. 3.4.21, E.C. 3.4.22, E.C. 3.4.23, E.C. 3.4.24. Example include, but are not limited to, trypsin, papain, pancreatin, pepsin (e.g., pepsin A, pepsin B), chymosin, cathepsin E, gastricsin, cathepsin D, phytepsin, cyprosin, cardosin A, cardosin B, nephentesin, neurosporapepsin, saccharopepsin, renin, plasmepsin, rhodorulapepsin, acrocyclindropepsin, pycnoporopepsin, physaropepsin, aspergillopepsin, penicillopepsin, rizopuspepsin, mucorpepsin, polyproppepsin, candidaparapsin, candidapepsin, yapsin 1, yapsin 2, yapsin 3, pseudomonaspepsin, xanzhornonpepsin, thermopsin, scytalidopepsin, aleurain, omptin, lysosomale, carboxypeptidase A, cathepsin A, lysosomale pro-X carboxypeptidase, asparaginyl endopeptidase, y-glutamylhydrolase, bacillus pepstatin insensitive acid endopeptidase, carboxypeptidase, and insulysin. Of these proteases, cardosin A, candidaparapsin, pseudomonapepsin, and pepsin (particularly pepsin A) are preferred for use. Corresponding substrates for proteases are various proteins and peptides. Proteases can be used in combination with zinc to enhance anti-plaque functions.

Carbohydrase enzymes act to break down or hydrolyze carbohydrates. Such enzymes are classified under the classification E.C. 3.2.1. Examples include, but are not limited to, dextranase, cellulase, amylase, α-glucosidase, β-glucosidase, lactase, invertase, amyloglucosidase, and lysozyme. Corresponding substrates for the carbohydrase enzymes include, but not limited to, dextran, cellulose, starch, oligosaccharides, beta-D-glucosides, lactose, sucrose, polysaccharides, and bacterial cell wall.

Lipase enzymes act to break down or hydrolyze fatty substances, e.g., fatty acids and fatty acid esters. Such enzymes are classified under classification E.C. 3.1.1 and E.C. 3.1.4. Examples include, but are not limited to, Lipase 4000, Lipase B, Lipase 448, gastric lipase, pancreatic lipase, and plant lipase. Corresponding substrates for lipase enzymes are various fats and oils.

Various combinations of enzymes and optional substrates can be used to enhance therapeutic functions. Examples include: combinations of various oxidoreductase enzymes and their corresponding substrates; combinations of glucose oxidase, glucose, and thiocyanate; and combinations of glucose oxidase/glucose dehydrogenase and glucose.

The enzyme preparations can be prepared to contain as high as 100% pure enzyme or may contain very low levels of enzyme, for example, 1% or less. Commercial enzyme preparations usually contain about 2 weight percent (wt-%) to about 80 wt-% of enzyme. Thus, the compositions of the present invention will include one or more enzymes and optionally one or more enzyme substrates taking into account both the activity of the enzyme preparation as well as its total amount. Generally, formulation will be based on activity, not on total weight of enzyme preparation. The level of enzyme used in the practice of this invention will depend on the enzymatic activity of the enzyme and the desired therapeutic effect.

Enzymes can be used in soluble form or immobilized form. An immobilized enzyme may be used to enhance enzymatic stability and reactivity. There are many methods available for immobilization including binding on prefabricated carrier materials and incorporating into in situ prepared carriers. Operative binding forces vary between weak multiple adsorptive interactions and single attachments through strong covalent binding. The appropriate methods depend on the enzyme structure and application. In general, enzymes can be immobilized by attachment to carriers through either chemical reaction or physical absorption and can be used in a variety of methods as described in W. Tischer, F. Wedekind, Topics in Current Chemistry, Vol. 200, Springer, Berlin Heidelberg, 1999. Alternatively, enzymes can be encapsulated within a membrane or liposome/micelle.

Polymerizable Components

The hardenable dental compositions of the present invention can also include a polymerizable component, thereby forming polymerizable compositions.

In certain embodiments, the compositions are photopolymerizable, i.e., the compositions contain a photoinitiator (i.e., a photoinitiator system) that upon irradiation with actinic radiation initiates the polymerization (or hardening) of the composition. Such photopolymerizable compositions can be free radically polymerizable or cationically polymerizable.

In certain embodiments, the compositions are chemically polymerizable, i.e., the compositions contain a chemical initiator (i.e., initiator system) that can polymerize, cure, or otherwise harden the composition without dependence on irradiation with actinic radiation. Such chemically polymerizable compositions are sometimes referred to as “self-cure” compositions and may include glass ionomer cements (e.g., conventional and resin-modified glass ionomer cements), redox cure systems, and combinations thereof.

Photopolymerizable Compositions

Suitable photopolymerizable compositions may include epoxy resins (which contain cationically active epoxy groups), vinyl ether resins (which contain cationically active vinyl ether groups), ethylenically unsaturated compounds (which contain free radically active unsaturated groups), and combinations thereof. Examples of useful ethylenically unsaturated compounds include acrylic acid esters, methacrylic acid esters, hydroxy-functional acrylic acid esters, hydroxy-functional methacrylic acid esters, and combinations thereof. Also suitable are polymerizable materials that contain both a cationically active functional group and a free radically active functional group in a single compound. Examples include epoxy-functional acrylates, epoxy-functional methacrylates, and combinations thereof.

Photopolymerizable compositions may include compounds having free radically active functional groups that may include monomers, oligomers, and polymers having one or more ethylenically unsaturated group. Suitable compounds contain at least one ethylenically unsaturated bond and are capable of undergoing addition polymerization. Such free radically polymerizable compounds include (meth)acrylates (i.e., acrylates and methacrylates) and (meth)acrylarides (i.e., acrylamides and methacrylamides), for example. Specific examples include mono-, di- or poly-acrylates and methacrylates such as methyl acrylate, methyl methacrylate, ethyl acrylate, isopropyl methacrylate, n-hexyl acrylate, stearyl acrylate, allyl acrylate, glycerol diacrylate, glycerol triacrylate, ethyleneglycol diacrylate, diethyleneglycol diacrylate, triethyleneglycol dimethacrylate, 1,3-propanediol diacrylate, 1,3-propanediol dimethacrylate, trimethylolpropane triacrylate, 1,2,4-butanetriol trimethacrylate, 1,4-cyclohexanediol diacrylate, pentaerythritol triacrylate, pentaerythritol tetraacrylate, pentaerythritol tetramethacrylate, sorbitol hexacrylate, bis[1-(2acryloxy)]-p-ethoxyphenyldimethylmethane, bis[1-(3-acryloxy-2-hydroxy)]-p-propoxyphenyldimethylmethane, and trishydroxyethyl-isocyanurate trimethacrylate; the bisacrylates and bis-methacrylates of polyethylene glycols of molecular weight 200-500, copolymerizable mixtures of acrylated monomers such as those in U.S. Pat. No. 4,652,274 (Boettcher et al.), and acrylated oligomers such as those of U.S. Pat. No. 4,642,126 (Zador et al.); and vinyl compounds such as styrene, diallyl phthalate, divinyl succinate, divinyl adipate and divinyl phthalate. Suitable ethylenically unsaturated compounds are also available from a wide variety of commercial sources, such as Sigma-Aldrich, St. Louis, Mo. and Rhom and Tech, Inc., Darmstadt, Germany. Other suitable free radically polymerizable compounds include siloxane-functional (meth)acrylates as disclosed, for example, in WO-00/38619 (Guggenberger et al.), WO-01/92271 (Weinmann et al.), WO-01/07444 (Guggenberger et al.), WO-00/42092 (Guggenberger et al.) and fluoropolymer-functional (meth)acrylates as disclosed, for example, in U.S. Pat. No. 5,076,844 (Fock et al.), U.S. Pat. No. 4,356,296 (Griffith et al.), EP-0373 384 (Wagenknecht et al.), EP-0201 031 (Reiners et al.), and EP-0201 778 (Reiners et al.). Mixtures of two or more free radically polymerizable compounds can be used if desired.

Photopolymerizable compositions may include compounds having cationically active functional groups such as cationically polymerizable epoxy resins. Such materials include organic compounds having an oxirane ring that is polymerizable by ring opening. These materials include monomeric epoxy compounds and epoxides of the polymeric type and can be aliphatic, cycloaliphatic, aromatic or heterocyclic. These compounds generally have, on the average, at least 1 polymerizable epoxy group per molecule, preferably at least about 1.5 and more preferably at least about 2 polymerizable epoxy groups per molecule. The polymeric epoxides include linear polymers having terminal epoxy groups (e.g., a diglycidyl ether of a polyoxyalkylene glycol), polymers having skeletal oxirane units (e.g., polybutadiene polyepoxide), and polymers having pendent epoxy groups (e.g., a glycidyl methacrylate polymer or copolymer). The epoxides may be pure compounds or may be mixtures of compounds containing one, two, or more epoxy groups per molecule. The “average” number of epoxy groups per molecule is determined by dividing the total number of epoxy groups in the epoxy-containing material by the total number of epoxy-containing molecules present.

These epoxy-containing materials may vary from low molecular weight monomeric materials to high molecular weight polymers and may vary greatly in the nature of their backbone and substituent groups. Illustrative of permissible substituent groups include halogens, ester groups, ethers, sulfonate groups, siloxane groups, nitro groups, phosphate groups, and the like. The molecular weight of the epoxy-containing materials may vary from about 58 to about 100,000 or more.

Suitable epoxy-containing materials useful in the present invention are listed in U.S. Pat. Nos. 6,187,836 (Oxman et al.) and 6,084,004 (Weinmann et al.).

Blends of various epoxy-containing materials are also contemplated. Examples of such blends include two or more weight average molecular weight distributions of epoxy-containing compounds, such as low molecular weight (below 200), intermediate molecular weight (about 200 to 10,000) and higher molecular weight (above about 10,000). Alternatively or additionally, the epoxy resin may contain a blend of epoxy-containing materials having different chemical natures, such as aliphatic and aromatic, or functionalities, such as polar and non-polar.

Other types of useful materials having cationically active functional groups include vinyl ethers, oxetanes, spiro-orthocarbonates, spiro-orthoesters, and the like.

If desired, both cationically active and free radically active functional groups may be contained in a single molecule. Such molecules may be obtained, for example, by reacting a di- or poly-epoxide with one or more equivalents of an ethylenically unsaturated carboxylic acid. An example of such a material is the reaction product of UVR-6105 (available from Union Carbide) with one equivalent of methacrylic acid. Commercially available materials having epoxy and free-radically active functionalities include the CYCLOMER series, such as CYCLOMER M-100, M-101, or A-200 available from Daicel Chemical, Japan, and EBECRYL-3605 available from Radcure Specialties, UCB Chemicals, Atlanta, Ga.

The cationically curable compositions may further include a hydroxyl-containing organic material. Suitable hydroxyl-containing materials may be any organic material having hydroxyl functionality of at least 1, and preferably at least 2. Preferably, the hydroxyl-containing material contains two or more primary or secondary aliphatic hydroxyl groups (i.e., the hydroxyl group is bonded directly to a non-aromatic carbon atom). The hydroxyl groups can be terminally situated, or they can be pendent from a polymer or copolymer. The molecular weight of the hydroxyl-containing organic material can vary from very low (e.g., 32) to very high (e.g., one million or more). Suitable hydroxyl-containing materials can have low molecular weights, i.e., from about 32 to about 200, intermediate molecular weights, i.e., from about 200 to about 10,000, or high molecular weights, i.e., above about 10,000. As used herein, all molecular weights are weight average molecular weights.

The hydroxyl-containing materials may be non-aromatic in nature or may contain aromatic functionality. The hydroxyl-containing material may optionally contain heteroatoms in the backbone of the molecule, such as nitrogen, oxygen, sulfur, and the like. The hydroxyl-containing material may, for example, be selected from naturally occurring or synthetically prepared cellulosic materials. The hydroxyl-containing material should be substantially free of groups which may be thermally or photolytically unstable; that is, the material should not decompose or liberate volatile components at temperatures below about 100° C. or in the presence of actinic light which may be encountered during the desired photopolymerization conditions for the polymerizable compositions.

Suitable hydroxyl-containing materials useful in the present invention are listed in U.S. Pat. No. 6,187,836 (Oxman et al.).

The amount of hydroxyl-containing organic material used in the polymerizable compositions may vary over broad ranges, depending upon factors such as the compatibility of the hydroxyl-containing material with the cationically and/or free radically polymerizable component, the equivalent weight and functionality of the hydroxyl-containing material, the physical properties desired in the final composition, the desired speed of polymerization, and the like.

Blends of various hydroxyl-containing materials may also be used. Examples of such blends include two or more molecular weight distributions of hydroxyl-containing compounds, such as low molecular weight (below about 200), intermediate molecular weight (about 200 to about 10,000) and higher molecular weight (above about 10,000). Alternatively, or additionally, the hydroxyl-containing material may contain a blend of hydroxyl-containing materials having different chemical natures, such as aliphatic and aromatic, or functionalities, such as polar and non-polar. As an additional example, one may use mixtures of two or more poly-functional hydroxy materials or one or more mono-functional hydroxy materials with poly-functional hydroxy materials.

The polymerizable material(s) may also contain hydroxyl groups and free radically active functional groups in a single molecule. Examples of such materials include hydroxyalkylacrylates and hydroxyalkylmethacrylates such as hydroxyethylacrylate, hydroxyethylmethacrylate; glycerol mono- or di-(meth)acrylate; trimethylolpropane mono- or di-(meth)acrylate, pentaerythritol mono-, di-, and tri-(meth)acrylate, sorbitol mono-, di-, tri-, tetra-, or penta-(meth)acrylate; and 2,2-bis[4-(2-hydroxy-3 methacryloxypropoxy)phenyl]propane.

The polymerizable material(s) may also contain hydroxyl groups and cationically active functional groups in a single molecule. An example is a single molecule that includes both hydroxyl groups and epoxy groups.

Suitable photoinitiators (i.e., photoinitiator systems that include one or more compounds) for polymerizing free radically photopolymerizable compositions include binary and tertiary systems. Typical tertiary photoinitiators include an iodonium salt, a photosensitizer, and an electron donor compound as described in U.S. Pat. No. 5,545,676 (Palazzotto et al.). Preferred iodonium salts are the diaryl iodonium salts, e.g., diphenyliodonium chloride, diphenyliodonium hexafluorophosphate, and diphenyliodonium tetrafluoroboarate. Preferred photosensitizers are monoketones and diketones that absorb some light within a range of about 450 nm to about 520 nm (preferably, about 450 nm to about 500 nm). More preferred compounds are alpha diketones that have some light absorption within a range of about 450 nm to about 520 nm (even more preferably, about 450 nm to about 500 nm). Preferred compounds are camphorquinone, benzil, furil, 3,3,6,6-tetramethylcyclohexanedione, phenanthraquinone and other cyclic alpha diketones. Most preferred is camphorquinone. Preferred electron donor compounds include substituted amines, e.g., ethyl dimethylaminobenzoate.

Suitable photoinitiators for polymerizing cationically photopolymerizable compositions include binary and tertiary systems. Typical tertiary photoinitiators include an iodonium salt, a photosensitizer, and an electron donor compound as described in U.S. Pat. Nos. 5,856,373 (Kaisaki et al.), 6,084,004 (Weinmann et al.), 6,187,833 (Oxman et al.), and 6,187,836 (Oxman et al.); and in U.S. Ser. No. 10/050218 (Dede et al.; filed Jan. 15, 2002). Preferred iodonium salts, photosensitizers, and electron donor compounds are as listed herein for photoinitiator systems for polymerizing free radically photopolymerizable compositions.

Other suitable photoinitiators for polymerizing free radically photopolymerizable compositions include the class of phosphine oxides that typically have a functional wavelength range of about 380 nm to about 1200 nm. Preferred phosphine oxide free radical initiators with a functional wavelength range of about 380 nm to about 450 nm are acyl and bisacyl phosphine oxides such as those described in U.S. Pat. Nos. 4,298,738 (Lechtken et al.), 4,324,744 (Lechtken et al.), 4,385,109 (Lechtken et al.), 4,710,523 (Lechtken et al.), and 4,737,593 (Ellrich et al.), 6,251,963 (Kohler et al.); and EP Application No. 0 173 567 A2 (Ying).

Commercially available phosphine oxide photoinitiators capable of free-radical initiation when irradiated at wavelength ranges of greater than about 380 nm to about 450 nm include bis(2,4,6-trimethylbenzoyl)phenyl phosphine oxide (IRGACURE 819, Ciba Specialty Chemicals, Tarrytown, N.Y.), bis(2,6-dimethoxybenzoyl)-(2,4,4-trimethylpentyl) phosphine oxide (CGI 403, Ciba Specialty Chemicals), a 25:75 mixture, by weight, of bis(2,6-dimethoxybenzoyl)-2,4,4-trimethylpentyl phosphine oxide and 2-hydroxy-2-methyl-1-phenylpropan-1-one (IRGACURE 1700, Ciba Specialty Chemicals), a 1:1 mixture, by weight, of bis(2,4,6-trimethylbenzoyl)phenyl phosphine oxide and 2-hydroxy-2-methyl-1-phenylpropane-1-one (DAROCUR 4265, Ciba Specialty Chemicals), and ethyl 2,4,6-trimethylbenzylphenyl phosphinate (LUCIRIN LR8893X, BASF Corp., Charlotte, N.C.).

Typically, the phosphine oxide initiator is present in the photopolymerizable composition in catalytically effective amounts, such as from about 0.1 wt-% to about 5.0 wt-%, based on the total weight of the composition.

Tertiary amine reducing agents may be used in combination with an acylphosphine oxide. Illustrative tertiary amines useful in the invention include ethyl 4-(N,N-dimethylamino)benzoate and N,N-dimethylaminoethyl methacrylate. When present, the amine reducing agent is present in the photopolymerizable composition in an amount from about 0.1 wt-% to about 5.0 wt-%, based on the total weight of the composition.

Chemically Polymerizable Compositions

The chemically polymerizable compositions may include glass ionomer cements such as conventional glass ionomer cements that typically employ as their main ingredients a homopolymer or copolymer of an ethylenically unsaturated carboxylic acid (e.g., poly acrylic acid, copoly (acrylic, itaconic acid), and the like), a fluoroaluminosilicate (“FAS”) glass, water, and a chelating agent such as tartaric acid. Conventional glass ionomers (i.e., glass ionomer cements) typically are supplied in powder/liquid formulations that are mixed just before use. The mixture will undergo self-hardening in the dark due to an ionic reaction between the acidic repeating units of the polycarboxylic acid and cations leached from the glass.

The glass ionomer cements may also include resin-modified glass ionomer (“RMGI”) cements. Like a conventional glass ionomer, an RMGI cement employs an FAS glass. However, the organic portion of an RMGI is different. In one type of RMGI, the polycarboxylic acid is modified to replace or end-cap some of the acidic repeating units with pendent curable groups and a photoinitiator is added to provide a second cure mechanism, e.g., as described in U.S. Pat. No. 5,130,347 (Mitra). Acrylate or methacrylate groups are usually employed as the pendant curable group. In another type of RMGI, the cement includes a polycarboxylic acid, an acrylate or methacrylate-functional monomer and a photoinitiator, e.g., as in Mathis et al., “Properties of a New Glass Ionomer/Composite Resin Hybrid Restorative”, Abstract No. 51, J. Dent Res., 66:113 (1987) and as in U.S. Pat. Nos. 5,063,257 (Akahane et al.), 5,520,725 (Kato et al.), 5,859,089 (Qian), 5,925,715 (Mitra) and 5,962,550 (Akahane et al.). In another type of RMGI, the cement may include a polycarboxylic acid, an acrylate or methacrylate-functional monomer, and a redox or other chemical cure system, e.g., as described in U.S. Pat. Nos. 5,154,762 (Mitra et al.), 5,520,725 (Kato et al.), and 5,871,360 (Kato). In another type of RMGI, the cement may include various monomer-containing or resin-containing components as described in U.S. Pat. Nos. 4,872,936 (Engelbrecht), 5,227,413 (Mitra), 5,367,002 (Huang et al.), and 5,965,632 (Orlowski). RMGI cements are preferably formulated as powder/liquid or paste/paste systems, and contain water as mixed and applied. The compositions are able to harden in the dark due to the ionic reaction between the acidic repeating units of the polycarboxylic acid and cations leached from the glass, and commercial RMGI products typically also cure on exposure of the cement to light from a dental curing lamp. RMGI cements that contain a redox cure system and that can be cured in the dark without the use of actinic radiation are described in U.S. patent Ser. No. 09/916399 (Mitra; Filed Jul. 27, 2001).

The chemically polymerizable compositions may include redox cure systems that include a polymerizable component (e.g., an ethylenically unsaturated polymerizable component) and redox agents. The redox agents may include an oxidizing agent and a reducing agent. Suitable polymerizable components, redox agents, optional acid-functional components, and optional fillers that are useful in the present invention are described in U.S. patent application Ser. No. 10/121,326 (Mitra et al.; Filed Apr. 12, 2002) and U.S. patent application Ser. No. 10/121,329 (Mitra et al.; Filed Apr. 12, 2002). Alternatively, the redox agents may include a free-radical initiator system containing enzymes as disclosed in Applicants' Assignees' copending application Ser. No. ______ (Attorney Docket No. 58071US002).

The reducing and oxidizing agents should react with or otherwise cooperate with one another to produce free-radicals capable of initiating polymerization of the resin system (e.g., the ethylenically unsaturated component). This type of cure is a dark reaction, that is, it is not dependent on the presence of light and can proceed in the absence of light. The reducing and oxidizing agents are preferably sufficiently shelf-stable and free of undesirable colorization to permit their storage and use under typical dental conditions. They should be sufficiently miscible with the resin system (and preferably water-soluble) to permit ready dissolution in (and discourage separation from) the other components of the polymerizable composition.

Useful reducing agents include ascorbic acid, ascorbic acid derivatives, and metal complexed ascorbic acid compounds as described in U.S. Pat. No. 5,501,727 (Wang et al.); amines, especially tertiary amines, such as 4-tert-butyl dimethylaniline; aromatic sulfinic salts, such as p-toluenesulfinic salts and benzenesulfinic salts; thioureas, such as 1-ethyl-2-thiourea, tetraethyl thiourea, tetramethyl thiourea, 1,1-dibutyl thiourea, and 1,3-dibutyl thiourea; and mixtures thereof. Other secondary reducing agents may include cobalt (II) chloride, ferrous chloride, ferrous sulfate, hydrazine, hydroxylamine (depending on the choice of oxidizing agent), salts of a dithionite or sulfite anion, and mixtures thereof. Preferably, the reducing agent is an amine.

Suitable oxidizing agents will also be familiar to those skilled in the art, and include but are not limited to persulfuric acid and salts thereof, such as sodium, potassium, ammonium, cesium, and alkyl ammonium salts. Additional oxidizing agents include peroxides such as benzoyl peroxides, hydroperoxides such as cumyl hydroperoxide, t-butyl hydroperoxide, and amyl hydroperoxide, as well as salts of transition metals such as cobalt (III) chloride and ferric chloride, cerium (IV) sulfate, perboric acid and salts thereof, permanganic acid and salts thereof, perphosphoric acid and salts thereof, and mixtures thereof.

It may be desirable to use more than one oxidizing agent or more than one reducing agent. Small quantities of transition metal compounds may also be added to accelerate the rate of redox cure. In some embodiments it may be preferred to include a secondary ionic salt to enhance the stability of the polymerizable composition as described in U.S. patent application File No. 57435US002 (Mitra et al.; Filed Apr. 12, 2002).

The reducing and oxidizing agents are present in amounts sufficient to permit an adequate free-radical reaction rate. This can be evaluated by combining all of the ingredients of the polymerizable composition except for the optional filler, and observing whether or not a hardened mass is obtained.

Preferably, the reducing agent is present in an amount of at least about 0.01 wt-%, and more preferably at least about 0.1 wt-%, based on the total weight (including water) of the components of the polymerizable composition. Preferably, the reducing agent is present in an amount of no greater than about 10 wt-%, and more preferably no greater than about 5 wt-%, based on the total weight (including water) of the components of the polymerizable composition.

Preferably, the oxidizing agent is present in an amount of at least about 0.01 wt-%, and more preferably at least about 0.10 wt-%, based on the total weight (including water) of the components of the polymerizable composition. Preferably, the oxidizing agent is present in an amount of no greater than about 10 wt-%, and more preferably no greater than about 5 wt-%, based on the total weight (including water) of the components of the polymerizable composition.

The reducing or oxidizing agents can be microencapsulated as described in U.S. Pat. No. 5,154,762 (Mitra et al.). This will generally enhance shelf stability of the polymerizable composition, and if necessary permit packaging the reducing and oxidizing agents together. For example, through appropriate selection of an encapsulant, the oxidizing and reducing agents can be combined with an acid-functional component and optional filler and kept in a storage-stable state. Likewise, through appropriate selection of a water-insoluble encapsulant, the reducing and oxidizing agents can be combined with an FAS glass and water and maintained in a storage-stable state.

A redox cure system can be combined with other cure systems, e.g., with a glass ionomer cement and with a photopolymerizable composition such as described U.S. Pat. No. 5,154,762 (Mitra et al.).

The hardenable compositions that utilize a redox cure system can be supplied in a variety of forms including two-part powder/liquid, paste/liquid, and paste/paste systems. Other forms employing multi-part combinations (i.e., combinations of two or more parts), each of which is in the form of a powder, liquid, gel, or paste are also possible. In a multi-part system, one part typically contains the reducing agent(s) and another part typically contains the oxidizing agent(s). Therefore, if the reducing agent is present in one part of the system, then the oxidizing agent is typically present in another part of the system. However, the reducing agent and oxidizing agent can be combined in the same part of the system through the use of the microencapsulation technique.

Fillers

The hardenable compositions of the present invention can also contain fillers. Fillers may be selected from one or more of a wide variety of materials suitable for incorporation in compositions used for dental applications, such as fillers currently used in dental restorative compositions, and the like.

The filler is preferably finely divided. The filler can have a unimodial or polymodial (e.g., bimodal) particle size distribution. Preferably, the maximum particle size (the largest dimension of a particle, typically, the diameter) of the filler is less than about 10 micrometers, and more preferably less than about 2.0 micrometers. Preferably, the average particle size of the filler is less than about 3.0 micrometers, and more preferably less than about 0.6 micrometer.

The filler can be an inorganic material. It can also be a crosslinked organic material that is insoluble in the resin system, and is optionally filled with inorganic filler. The filler should in any event be nontoxic and suitable for use in the mouth. The filler can be radiopaque or radiolucent. The filler is also substantially insoluble in water.

Examples of suitable inorganic fillers are naturally occurring or synthetic materials including, but not limited to: quartz; nitrides (e.g., silicon nitride); glasses derived from, for example, Ce, Sb, Sn, Ba, Zn, and Al; feldspar; borosilicate glass; kaolin; talc; titania; low Mohs hardness fillers such as those described in U.S. Pat. No. 4,695,251 (Randklev); and submicron silica particles (e.g., pyrogenic silicas such as those available under the trade designations AEROSIL, including “OX 50,” “130,” “150” and “200” silicas from Degussa Corp., Akron, Ohio and CAB-O-SIL M5 silica from Cabot Corp., Tuscola, Ill.). Examples of suitable organic filler particles include filled or unfilled pulverized polycarbonates, polyepoxides, and the like.

Preferred non-acid-reactive filler particles are quartz, submicron silica, and non-vitreous microparticles of the type described in U.S. Pat. No. 4,503,169 (Randklev). Mixtures of these non-acid-reactive fillers are also contemplated, as well as combination fillers made from organic and inorganic materials.

The surface of the filler particles can also be treated with a coupling agent in order to enhance the bond between the filler and the resin. The use of suitable coupling agents include gamma-methacryloxypropyltrimethoxysilane, gamma-mercaptopropyltriethoxysilane, gamma-aminopropyltrimethoxysilane, and the like.

The filler can also be an acid-reactive filler. An acid-reactive filler is typically used in combination with an acid-functional resin component, and may or may not be used in combination with a nonreactive filler. The acid-reactive filler can, if desired, also possess the property of releasing fluoride. Suitable acid-reactive fillers include metal oxides, glasses, and metal salts. Preferred metal oxides include barium oxide, calcium oxide, magnesium oxide, and zinc oxide. Preferred glasses include borate glasses, phosphate glasses, and fluoroaluminosilicate (“FAS”) glasses. FAS glasses are particularly preferred. The FAS glass preferably contains sufficient elutable cations so that a hardened dental composition will form when the glass is mixed with the components of the hardenable composition. The glass also preferably contains sufficient elutable fluoride ions so that the hardened composition will have cariostatic properties. The glass can be made from a melt containing fluoride, alumina, and other glass-forming ingredients using techniques familiar to those skilled in the FAS glassmaking art. The FAS glass preferably is in the form of particles that are sufficiently finely divided so that they can conveniently be mixed with the other cement components and will perform well when the resulting mixture is used in the mouth.

Preferably, the average particle size (typically, diameter) for the FAS glass is no greater than about 10 micrometers, and more preferably no greater than about 5 micrometers as measured using, for example, a sedimentation analyzer. Suitable FAS glasses will be familiar to those skilled in the art, and are available from a wide variety of commercial sources, and many are found in currently available glass ionomer cements such as those commercially available under the trade designations VITREMER, VITREBOND, RELY X LUTING CEMENT and KETAC-FIL (3M ESPE Dental Products, St. Paul, Minn.), FUJI II, GC FUJI LC and FUJI IX (G-C Dental Industrial Corp., Tokyo, Japan) and CHEMFIL Superior (Dentsply International, York, Pa.). Mixtures of fillers can be used if desired.

The FAS glass can optionally be subjected to a surface treatment. Suitable surface treatments include, but are not limited to, acid washing (e.g., treatment with a phosphoric acid), treatment with a phosphate, treatment with a chelating agent such as tartaric acid, and treatment with a silane or an acidic or basic silanol solution. Desirably the pH of the treating solution or the treated glass is adjusted to neutral or near-neutral, as this can increase storage stability of the hardenable composition.

In certain compositions mixtures of acid-reactive and non-acid-reactive fillers can be used either in the same part or in different parts.

Other suitable fillers are disclosed in U.S. Pat. No. 6,387,981 (Zhang et al.) as well as International Publication Nos. WO 01/30304 (Wu et al.), WO 01/30305 (Zhang et al.), WO 01/30306 (Windisch et al.), and WO 01/30307 (Zhang et al.). Other suitable fillers are described in references cited within these publications.

U.S. Pat. No. 6,306,926 (Bretscher et al.) discloses a number of radiopacifying fillers that can be used in both free radically polymerizable compositions, cationically polymerizable compositions, and hybrid compositions featuring both free radically and cationically polymerizable components. They are particularly advantageous for use in cationically polymerizable compositions. One such filler is a melt-derived filler that includes 5-25% by weight aluminum oxide, 10-35% by weight boron oxide, 15-50% by weight lanthanum oxide, and 20-50% by weight silicon oxide. Another filler is a melt-derived filler that includes 10-30% by weight aluminum oxide, 10-40% by weight boron oxide, 20-50% by weight silicon oxide, and 15-40% by weight tantalum oxide. A third filler is a melt-derived filler that includes 5-30% by weight aluminum oxide, 5-40% by weight boron oxide, 0-15% by weight lanthanum oxide, 25-55% by weight silicon oxide, and 10-40% by weight zinc oxide. A fourth filler is a melt-derived filler that includes 15-30% by weight aluminum oxide, 15-30% by weight boron oxide, 20-50% by weight silicon oxide, and 15-40% by weight ytterbium oxide. A fifth filler is in the form of non vitreous microparticles prepared by a sol-gel method in which an aqueous or organic dispersion or sol of amorphous silicon oxide is mixed with an aqueous or organic dispersion, sol, or solution of a radiopacifying metal oxide, or precursor organic or compound. A sixth filler is in the form of non-vitreous microparticles prepared by a sol-gel method in which an aqueous or organic dispersion or sol of amorphous silicon oxide is mixed with an aqueous or organic dispersion, sol, or solution of a radiopacifying metal oxide, or precursor organic or inorganic compound.

Optional Additives

The compositions of the invention can optionally contain water. The water can be distilled, deionized, or plain tap water. Generally, deionized water is preferred.

If present, the amount of water should be sufficient to provide adequate handling and mixing properties and to permit the transport of ions, particularly in the filler-acid reaction. Preferably, water represents at least about 1 wt-%, and more preferably at least about 5 wt-%, of the total weight of ingredients used to form the hardenable composition. Preferably, water represents no greater than about 75 wt-%, and more preferably no greater than about 50 wt-%, of the total weight of ingredients used to form the hardenable composition.

Optionally, the hardenable compositions also may contain solvents (e.g., alcohols) or diluents other than water. These cosolvents are at least partially water miscible and include, for example, tetrahydrofuran, acetone, dioxane, dimethyl formamide, dimethyl sulfoxide, ethanol, methanol, propanol, isopropanol, butanol, isobutanol, ethylene glycol, ethylene glycol monomethyl ether, and propylene glycol.

The amount of cosolvent should be sufficient to provide sufficient dissolution and reactivity of the composition components. Preferably, the cosolvent represents at least about 1 wt-%, and more preferably at least about 5 wt-%, of the total weight of ingredients used to form the hardenable composition. Preferably, the cosolvent represents no greater than about 75 wt-%, and more preferably no greater than about 50 wt-%, of the total weight of ingredients used to form the hardenable composition.

If desired, the hardenable composition of the invention can contain additives such as pigments, inhibitors, accelerators, viscosity modifiers, surfactants, and other ingredients that will be apparent to those skilled in the art. Additionally, medicaments (other than the therapeutic enzyme) can be optionally added to the dental compositions. Examples include anti-inflammatory agents, antimicrobial agents, whitening agents, and the like, of the type often used in dental compositions. The selection and amount of any one such additive can be selected by one of skill in the art to accomplish the desired result without undue experimentation.

Preparation and Use of the Compositions

The hardenable dental compositions of the present invention can be prepared by combining a therapeutic enzyme with a polymerizable component using conventional mixing techniques. The resulting composition may optionally contain fillers, water, co-solvents, and other additives as described herein. In some embodiments, as described herein, an enzyme substrate may be added to the composition. In use, the compositions may contain a photoinitiator and be hardened by photoinitiation, or may be hardened by chemical polymerization such as a redox cure system in which the composition contains a free-radical initiator system, e.g., including an oxidizing agent and a reducing agent. Alternatively, the hardenable composition may contain different initiator systems, such that the composition can be both a photpolymerizable and a chemically polymerizable composition.

The hardenable compositions of the invention can be supplied in a variety of forms including one-part systems and multi-part systems, e.g., two-part powder/liquid, paste/liquid, and paste/paste systems. Other forms employing multi-part combinations (i.e., combinations of two or more parts), each of which is in the form of a powder, liquid, gel, or paste are also possible. In a redox multi-part system, one part typically contains the oxidizing agent and another part typically contains the reducing agent. In multi-part systems containing an enzyme substrate, one part typically contains the therapeutic enzyme and another part typically contains the enzyme substrate.

The components of the hardenable composition can be included in a kit, where the contents of the composition are packaged, as described below, to allow for storage of the components until they are needed.

When used as a dental composition, the components of the hardenable compositions can be mixed and clinically applied using conventional techniques. A curing light is generally required for the initiation of photopolymerizable compositions. The compositions can be in the form of composites or restoratives that adhere very well to dentin and/or enamel. Optionally, a primer layer can be used on the tooth tissue on which the hardenable composition is used. The compositions, e.g., containing a FAS glass or other fluoride releasing material, can also provide very good long-term fluoride release. Some embodiments of the invention may provide glass ionomer cements or adhesives that can be cured in bulk without the application of light or other external curing energy, do not require a pre-treatment, have improved physical properties including improved flexural strength, and have high fluoride release for cariostatic effect.

The compositions of the invention are particularly well adapted for use in the form of a wide variety of dental materials, which may be filled or unfilled. They can be used in sealants or adhesives, which are lightly filled composites (up to about 25 wt-% filler, based on the total weight of the composition) or unfilled compositions that are cured after being dispensed adjacent to a tooth (i.e., placing a dental material in temporary or permanent bonding or touching contact with a tooth). They can be used in cements, which are typically filled compositions (preferably containing greater than about 25 wt-% filler and up to about 60 wt-% filler). They can also be used in restoratives, which include composites that are polymerized after being disposed adjacent to a tooth, such as filling materials. They can also be used in prostheses that are shaped and polymerized for final use (e.g., as a crown, bridge, veneer, inlay, onlay, or the like), before being disposed adjacent to a tooth. Such preformed articles can be ground or otherwise formed into a custom-fitted shape by the dentist or other user. Although the hardened dental material can be any of a wide variety of materials that are prepared from polymerizable materials, preferably, the hardened dental material is not a surface pre-treatment material (e.g., etchant, primer, bonding agent). Rather, preferably, the hardened dental material is a restorative (e.g., composite, filling material or prosthesis), cement, sealant, coating, or orthodontic adhesive.

The compositions have utility in clinical applications where cure of conventional light-curable cement may be difficult to achieve. Such applications include, but are not limited to, deep restorations, large crown build-ups, endodontic restorations, attachment of orthodontic brackets (including pre-coated brackets, where, for example, a paste portion could be pre-applied to the bracket and a liquid portion could later be brushed onto a tooth), bands, buccal tubes, and other devices, luting of metallic crowns or other light-impermeable prosthetic devices to teeth, and other restorative applications in inaccessible areas of the mouth.

The hardenable dental compositions in the form of the dental materials as described above or in alternative forms can be used to provide a therapeutic enzyme to a subject's mouth for an extended period of time, e.g., at least 1 day, alternatively at least 7 days, and in some embodiments at least 30 days, and often at least 90 days. Depending on the therapeutic enzyme and the optional additives utilized, a variety of desirable end-use results can be achieved. Examples include the use of glucose oxidase to catalyze the reaction between water, oxygen and glucose in the mouth and thereby release antibacterial agents; and hydrolase enzymes (e.g., such as DXAMase from Lifenza, Seoul, Korea) to catalyze the decomposition of harmful carbohydrates, proteins, lipids, and/or bacteriocidal substrates in the oral plaque and saliva.

### EXAMPLES

Objects and advantages of this invention are further illustrated by the following examples, but the particular materials and amounts thereof recited in these examples, as well as other conditions and details, should not be construed to unduly limit this invention. Unless otherwise indicated, all parts and percentages are on a weight basis, all water is deionized water, and all molecular weights are weight average molecular weight.

Abbreviations/Definitions

| | |
|---|---|
| o-dianisidine | 3,3′-Dimethoxybenzidine (Sigma-Aldrich, St. Louis, MO) |
| Buffer | Sodium acetate buffer (0.05 M; pH 5.1) stored at 2-8° C. |
| GOx | Glucose Oxidase X-S (50,000 Units in 0.21 gram (g)) |
| | (Sigma-Aldrich) |
| Peroxidase | Peroxidase VI-A (Sigma-Aldrich) |
| Peroxidase-S | Peroxidase Assay Solution prepared by dissolving |
| | Peroxidase (0.76 milligram (mg); 1000 Units) and o- |
| | dianisidine (4 mg) in Buffer (enough to make 40 milliliter |
| | (ml) of total Solution). Stored at 2-8° C. |
| Glucose-S | Glucose Standard Solution (100 milligram per deciliter |
| | (mg/dl)) (Sigma-Aldrich) |
| FILTEK | FILTEK Supreme Restorative (3M ESPE, 3M Co., St. Paul, |
| | MN) |
| CLINPRO | CLINPRO Sealant (3M ESPE) |
| Composition | Orthodontic adhesive prepared as described for Example 10 |
| A | in U.S. Pat. application Ser. No. 10/126,505, filed on |
| | Apr. 18, 2002 |
| APC II | APC II Orthodontic Adhesive (3M Unitek, 3M Co., St. |
| | Paul, MN) |
| VITREMER | VITREMER Luting Cement (3M ESPE) |
| SINGLE | SINGLE BOND Adhesive (3M ESPE) |
| BOND | |

### Test Methods

Enzymatic Activity Assay

A solution of Glucose-S (0.05 ml) and water (0.95 ml) was added to a first container and water (1 ml) was added to a second container (Control). A sample of Peroxidase-S (2 ml) was added to each container and the contents mixed well. A sample disc containing GOx in a hardened composition was added to the solution in each container and the exact time recorded. The containers were placed in a 37° C. oven for 30 minutes, the discs removed, and 12 Normal (12N) sulfuric acid (2.0 ml) added. The absorbance of the solution in each container was measured at peak between a wavelength range of 523 and 532 nanometers (nm) using a spectrophotometer. (HP 8452A Diode Array Spectrophotometer) (This wavelength range is the absorbance range of oxidized o-dianisidine that would be formed from o-dianisidine in the presence of hydrogen peroxide that in turn would be present as a by-product from the reaction of glucose and GOx in the presence of water and oxygen.) Absorbance values are reported as Absorbance Units (AU) per gram of GOx with higher values indicative of greater levels of enzymatic activity. Results are also reported as Relative Activity that is equal to the Example (with glucose) Absorbance Value less the Control Example (without glucose) Absorbance Value.

### Examples 1 to 7

A series of materials (Examples 1 to 7) were prepared by mixing at room temperature (approximately 23° C.) GOx with a variety of hardenable compositions. The amount of GOx and the identification and amounts of the compositions utilized are listed in Table 1. In the case of VITREMER (two-part system containing a liquid part and a powder part), one example (Example 5) was prepared by mixing the GOx first with the liquid part and then combining with the powder part; whereas another example (Example 6) was prepared by mixing the GOx first with the powder part and then combining with the liquid part. In both cases the weight ratio of liquid to powder was 1.6. Two solid disc samples were prepared from each of the resulting mixed compositions as follows:

For light-cured compositions (all compositions, except for VITREMER) a sample of the mixed composition was placed in a disc-shaped cavity (5.1-centimeter (cm) diameter×1-millimeter (mm) thick) of a steel mold, cured for 2 minutes with a dental curing light, and the resulting hard disc removed from the mold. The weight of the sample was recorded before and after curing.

For the VITREMER compositions, a sample of the mixed composition (mixing time of about 40 seconds) was placed in the disc-shaped cavity, allowed to self-cure for 3-4 minutes at room temperature, and the resulting hard disc removed from the mold. The weight of the sample was recorded before and after curing.

### Evaluations and Results of Examples 1-7

The enzyme activities of the hardened discs (Examples 1-7) were evaluated according to the Enzymatic Activity Assay Test Method described herein. The results of the evaluations are provided in Table 1 and are compared with the results of the corresponding control samples (without added glucose).

The results in Table 1 show that the enzymatic activity was greatest for the orthodontic adhesives (“CESSNA” and ATC II) and the dental restorative (FILTEK).

TABLE 1

| | | | | | Relative |
|---|---|---|---|---|---|
| | | Compositions | | | Activity |
| | GOx | | Amount | Absorbance | (Ex. - |
| Example | (mg) | Type | (g) | (AU/g GOx) | Control) |
| 1 | 10 | FILTEK | 5 | 177.8 | 162.6 |
| Control 1 | 10 | FILTEK | 5 | 15.2 | |
| 2 | 10 | CLINPRO | 5 | 120 | 101.2 |
| Control 2 | 10 | CLINPRO | 5 | 18.8 | |
| 3 | 10 | Composition A | 5 | 229.6 | 210.2 |
| Control 3 | 10 | Composition A | 5 | 19.4 | |
| 4 | 10 | APC II | 5 | 201.5 | 182.4 |
| Control 4 | 10 | APC II | 5 | 19.1 | |
| 5 | 6.2 | VITREMER | 1.9 | 241.1 | 150.5 |
| | | (Liquid First) | | | |
| Control 5 | 6.2 | VITREMER | 1.9 | 90.6 | |
| | | (Liquid First) | | | |
| 6 | 3.8 | VITREMER | 3.1 | 107.7 | 56.1 |
| | | (Powder First) | | | |
| Control 6 | 3.8 | VITREMER | 3.1 | 51.6 | |
| | | (Powder First) | | | |
| 7 | 10 | SINGLE BOND | 5 | 33.1 | −5.1 |
| Control 7 | 10 | SINGLE BOND | 5 | 38.2 | |

### Example 8

One (1) mg pepsin (manufactured by Sigma) was added to 1 g of the powder component of the commercially available standard glass ionomer cement Ketac Molar manufactured by 3M ESPE, Seefeld, Germany), after which the mixture was mixed thoroughly. In accordance with the instructions for the use of Ketac Molar, 1 g each of Ketac Molar with and without pepsin was hardened in 24× microtiter plates.

First, the samples were coated with a 0.5 ml disodium hydrogen phosphate buffer (50 nM, pH 7.0) containing 25 micrograms (μg) fluorescence-marking casein (manufactured by Molecular Probes). After 30 minutes, the remaining solution was moved to a fluorescence vessel with an eye dropper and the fluorescence measured at an excitation of 480 nm and an emission of 510 nm. No significant increase in fluorescence could be measured, which indicates that the pepsin exhibits no catalytic activity at this physiological pH (7.0).

In a second experiment, the Ketac Molar samples with and without pepsin were coated with 0.5 ml lactic acid solution (2 mM, pH 3.8) containing 25 μg fluorescence-marking casein (manufactured by Molecular Probes). After 30 minutes, the remaining solution was moved to a fluorescence vessel with an eye dropper and the fluorescence measured at an excitation of 480 nm and an emission of 510 nm. The Ketac Molar sample without pepsin displayed no significant increase in fluorescence, while the Ketac Molar samples with pepsin displayed a significant fluorescence increase. The pH change to 3.8 triggered the catalytic activity of pepsin.

The bactericidal effect of the pepsin in the Ketac Molar was proven using the LIVE/DEAD BacLight Bacterial Viability Kit commercially available from Molecular Probes, Leiden, Netherlands. This was documented by adding one drop of a lactobacillus paracasei culture solution to Ketac Molar samples with and without pepsin. Examination under the fluorescence microscope (Zeiss Axioplan 2) showed up to 85% fewer living bacteria on the Ketac Molar samples with pepsin than on the Ketac Molar samples without pepsin.

The complete disclosures of the patents, patent documents, and publications cited herein are incorporated by reference in their entirety as if each were individually incorporated. Various modifications and alterations to this invention will become apparent to those skilled in the art without departing from the scope and spirit of this invention. It should be understood that this invention is not intended to be unduly limited by the illustrative embodiments and examples set forth herein and that such examples and embodiments are presented by way of example only with the scope of the invention intended to be limited only by the claims set forth herein as follows.

What is claimed is:

1. A hardenable dental composition comprising a polymerizable component and a therapeutic enzyme mixed within the polymerizable component, wherein upon hardening the polymerizable component to form a hardened dental material having a therapeutic enzyme mixed therein, the hardened dental material with the enzyme mixed therein is in contact with saliva in a subject's mouth for at least 1 day.

2. The hardenable dental composition of claim 1 wherein the hardened dental material with the enzyme mixed therein is in contact with saliva in a subject's mouth for at least 7 days.

3. The hardenable dental composition of claim 1 wherein the composition is photopolymerizable.

4. The hardenable dental composition of claim 3 wherein the polymerizable component is selected from the group consisting of epoxy resins, vinyl ether resins, ethylenically unsaturated compounds, and combinations thereof.

5. The hardenable dental composition of claim 1 wherein the composition is chemically polymerizable.

6. The hardenable dental composition of claim 5 wherein the polymerizable component comprises an ethylenically unsaturated compound, a glass ionomer cement, or a combination thereof.

7. The hardenable dental composition of claim 1 further comprising a filler.

8. The hardenable dental composition of claim 1 wherein the enzyme is an oxidoreductase.

9. The hardenable dental composition of claim 8 wherein the enzyme is an oxidase, a peroxidase, laccase, or mixture thereof.

10. The hardenable dental composition of claim 1 wherein the enzyme is a hydrolase.

11. The hardenable dental composition of claim 10 wherein the enzyme is a protease, a carbohydrase, a lipase, or a mixture thereof.

12. The hardenable dental composition of claim 11 wherein the enzyme is a protease.

13. The hardenable dental composition of claim 12 wherein the enzyme is a pepsin.

14. The hardenable dental composition of claim 1 further comprising an enzyme substrate.

15. A hardenable dental composition comprising a polymerizable component and a therapeutic enzyme mixed within the polymerizable component;
wherein upon hardening the polymerizable component to form a hardened dental material having a therapeutic enzyme mixed therein, the hardened dental material with the enzyme mixed therein is in contact with saliva in a subject's mouth for at least 1 day;
wherein the therapeutic enzyme is selected from the group consisting of an oxidoreductase, a hydrolase, and combinations thereof; and
wherein the polymerizable component is selected from the group consisting of epoxy resins, vinyl ether resins, ethylenically unsaturated compounds, glass ionomer cements, and combinations thereof.

16. The hardenable dental composition of claim 15 wherein the enzyme is an oxidoreductase.

17. The hardenable dental composition of claim 16 wherein the enzyme is an oxidase, a peroxidase, laccase, or mixture thereof.

18. The hardenable dental composition of claim 15 wherein the enzyme is a hydrolase.

19. The hardenable dental composition of claim 18 wherein the enzyme is a protease, a carbohydrase, a lipase, or a mixture thereof.

20. The hardenable dental composition of claim 19 wherein the enzyme is a protease.

21. The hardenable dental composition of claim 20 wherein the enzyme is a pepsin.

22. The hardenable dental composition of claim 15 further comprising an enzyme substrate.

23. The hardenable dental composition of claim 15 wherein the hardened dental material with the enzyme mixed therein is in contact with saliva in a subject's mouth for at least 7 days.

24. A hardenable dental composition comprising a polymerizable component and a therapeutic enzyme mixed within the polymerizable component;
wherein upon hardening the polymerizable component to form a hardened dental material having a therapeutic enzyme mixed therein, the hardened dental material with the enzyme mixed therein is in contact with saliva in a subject's mouth for at least 1 day;
wherein the therapeutic enzyme is selected from the group consisting of oxidases, peroxidases, laccases, proteases, carbohydrases, lipases, and combinations thereof; and
wherein the polymerizable component is selected from the group consisting of (meth)acrylates, (meth)acrylamides, and combinations thereof.

25. The hardenable dental composition of claim 24 wherein the enzyme is an oxidase, a peroxidase, laccase, or mixture thereof.

26. The hardenable dental composition of claim 24 wherein the enzyme is a protease, a carbohydrase, a lipase, or a mixture thereof.

27. The hardenable dental composition of claim 26 wherein the enzyme is a protease.

28. The hardenable dental composition of claim 27 wherein the enzyme is a pepsin.

29. The hardenable dental composition of claim 24 further comprising an enzyme substrate.

30. The hardenable dental composition of claim 24 wherein the hardened dental material with the enzyme mixed therein is in contact with saliva in a subject's mouth for at least 7 days.

31. A method of delivering a therapeutic enzyme to a subject's mouth to improve the oral health of the subject, the method comprising:
providing a hardenable dental composition comprising a polymerizable component and a therapeutic enzyme mixed within the polymerizable component;
placing the hardenable dental composition in the mouth of the subject; and
hardening the composition to form a hardened dental material having a therapeutic enzyme mixed therein, the hardened material with the enzyme mixed therein is in contact with saliva in a subject's mouth for at least 1 day.

32. The method of claim 31 wherein the hardened dental material with the enzyme mixed therein is in contact with saliva in a subject's mouth for at least 7 days.

33. The method of claim 31 wherein the composition is photopolymerizable.

34. The method of claim 33 wherein the polymerizable component is selected from the group consisting of epoxy resins, vinyl ether resins, ethylenically unsaturated compounds, and combinations thereof.

35. The method of claim 31 wherein the composition is chemically polymerizable.

36. The method of claim 35 wherein the polymerizable component comprises an ethylenically unsaturated compound, a glass ionomer cement, or a combination thereof.

37. The method of claim 31 wherein the hardenable dental composition further comprises a filler.

38. The method of claim 31 wherein the enzyme is an oxidoreductase.

39. The method of claim 38 wherein the enzyme is an oxidase, a peroxidase, laccase, or mixture thereof.

40. The method of claim 31 wherein the enzyme is a hydrolase.

41. The method of claim 40 wherein the enzyme is a protease, a carbohydrase, a lipase, or a mixture thereof.

42. The method of claim 41 wherein the enzyme is a protease.

43. The method of claim 42 wherein the enzyme is a pepsin.

44. The method of claim 31 wherein the hardenable dental composition further comprises an enzyme substrate.

45. The method of claim 31 wherein the hardened dental material is a restorative, sealant, cement, coating, or orthodontic adhesive.
